(19) United States
(12) Plant Patent Application Publication
(10) Pub. No.: US 2026/0240060 P1
(43) Pub. Date: Aug. 13, 2026

(54) STRAWBERRY PLANT NAMED 'CULTA-T3L'

(50) ;

(71) Applicant: Culta, Inc., Tokyo (JP)

(72) Inventors: Shuhei NOAKI, Tokyo (JP); Ayako YAMAGUCHI, Tokyo (JP); Jiayi LIU, Tokyo (JP)

(21) Appl. No.: 19/466,904

(22) Filed: Feb. 2, 2026

(30) Foreign Application Priority Data
Feb. 6, 2025 (JP) 37874

(51) Int. Cl. A01H 6/74; A01H 5/08

(52) U.S. Cl. CPC A01H 6/7409; A01H 5/08
USPC PLT/208

(57) ABSTRACT

This invention relates to a new and distinct variety of strawberry plant named ‘CULTA-T3L’. This new strawberry plant named ‘CULTA-T3L’ is primarily adapted to the growing conditions in central Japan, and is primarily characterized by its high sugar content, high fruit firmness, and 5 vigorous stolon production.

Latin name of the genus and species of the plant claimed: Fragaria x ananassa:

Variety denomination: ‘CULTA-T3L’:

### CROSS-REFERENCE TO RELATED APPLICATIONS

This application claims priority to Japanese Variety Registration Application No. 37874 filed Feb. 6, 2025, which is incorporated by reference herein as if set forth in its entirety.

### DESCRIPTION

### Background of the Invention

The present invention relates to a new and distinct strawberry variety named ‘CULTA-T3L’. This new variety is a result of a controlled cross made in 2021 in an ongoing breeding program. The maternal and paternal lines are unnamed. The variety is botanically known as Fragaria x ananassa.

The seedling resulting from the aforementioned cross was selected from a controlled breeding plot in Tokyo, Japan from October 2022 to March 2023. After its selection, the new variety was asexually propagated by stolons in Saitama, Japan. The new variety was tested extensively over the next several years in fruiting fields in Tokyo and Saitama, Japan. This propagation has demonstrated that the combination of traits disclosed herein as characterizing the new variety are fixed and remain true-to-type through successive generations of asexual reproduction.

### BRIEF SUMMARY OF THE INVENTION

‘CULTA-T3L’ is primarily adapted to the climate and growing conditions of the central coast of Japan. The following traits have been repeatedly observed and are determined to be unique characteristics of ‘CULTA-T3L’, which in combination distinguish this strawberry plant as a new and distinct variety:
1. Fruit has a high sugar content with a representative average BRIX of approximately 12.8% when measured as described herein;
2. Fruit has extremely high firmness with a representative average firmness of approximately 0.51 kg when measured as described herein;
3. Plants have vigorous stolon production.
4. Plants exhibit strong vegetative growth.

The strawberry varieties that are believed to be most closely related to the new variety ‘CULTA-T3L’ are ‘Benihoppe’ and ‘Akihime’. In side-by-side comparisons to the similar strawberry varieties Benihoppe' and ‘Akihime’, ‘CULTA-T3L’ differs by the following combination of characteristics as described in Table 1.

TABLE 1

| Name of | | State of | State of |
|---|---|---|---|
| similar | | similar | ‘CULTA- |
| variety | Characteristic | variety | T3L’ |
| ‘Benihoppe’ | Number of | Many | Many to very |
| JVPA 10371 | stolons | | many |
| ‘Akihime’ | Fruit: firmness | Soft to | Very firm |
| JVPA 2991 | | medium | |
| ‘Akihime’ | Fruit: cavity | Absent or | Large |
| | | small | |

### BRIEF DESCRIPTION OF THE PHOTOGRAPHS

The accompanying color photographs illustrate the overall appearance of typical specimens of the new strawberry variety ‘CULTA-T3L’ at various stages of development, as true as it is reasonably possible with color reproductions of this type. Color in the photographs may differ slightly from the color value cited in the botanical descriptions which accurately describe the color of ‘CULTA-T3L’. The depicted plant and plant parts of the new strawberry variety are approximately 5-6 months old. The photographs were taken near Saitama, Japan.

FIG. 1 shows fruiting field characteristics of ‘CULTA-T3L’, taken in the month of January 2025.

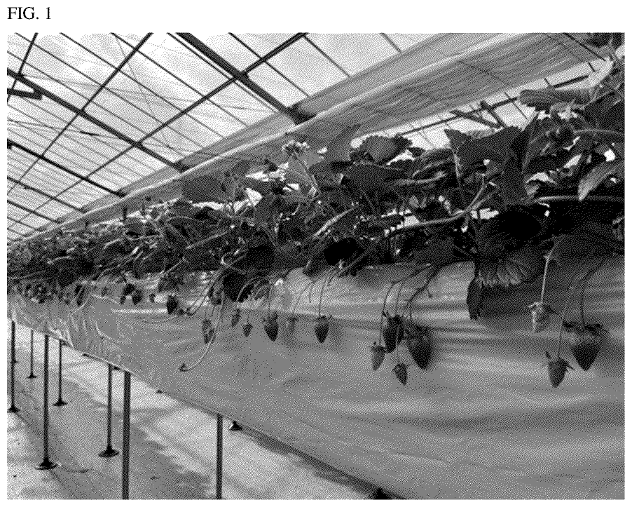

FIG. 2 shows a close up of fruiting field characteristics of ‘CULTA-T3L’, taken in the month of January 2025.

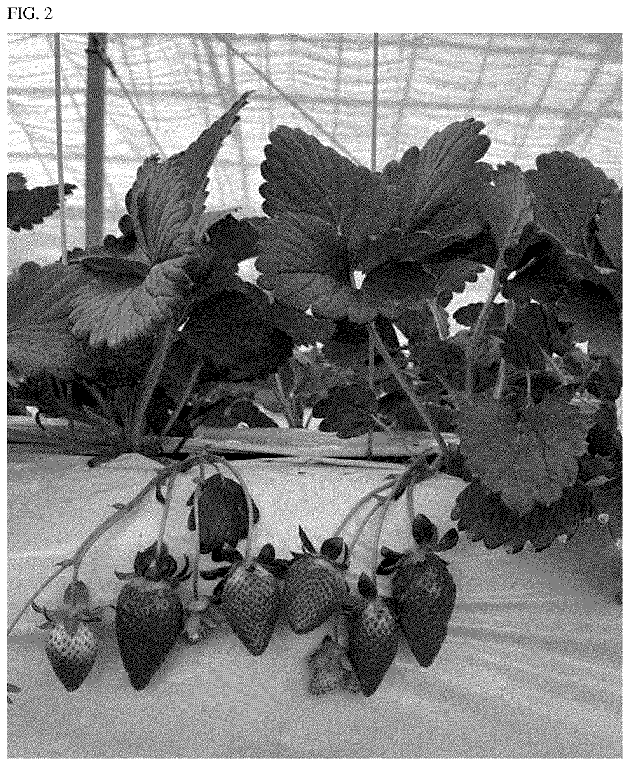

FIG. 3 shows mature fruit characteristics of ‘CULTA-T3L’, taken in the month of January 2025.

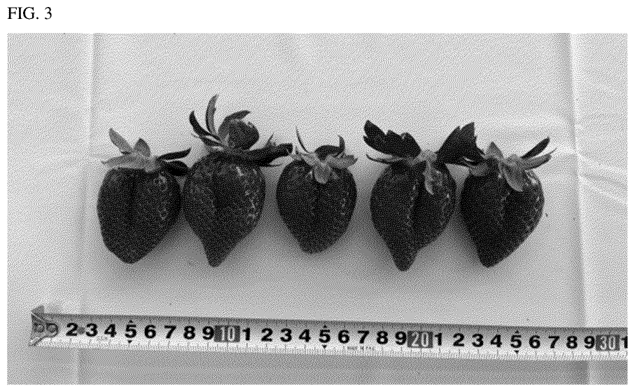

FIG. 4 shows mature fruit characteristics of ‘CULTA-T3L’, taken in the month of January 2025.

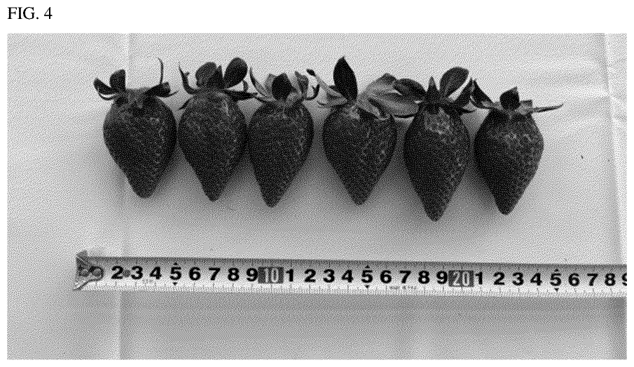

FIG. 5 shows characteristics of the top of the mature fruit of ‘CULTA-T3L’, taken in the month of January 2025.

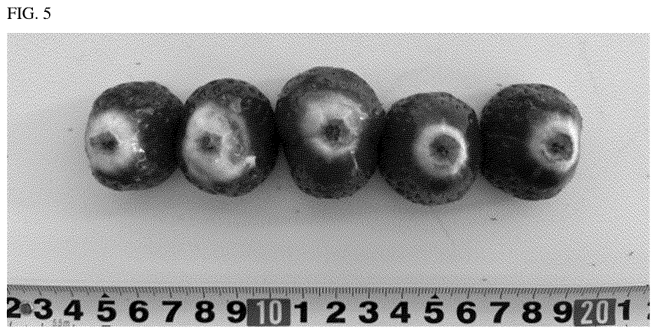

FIG. 6 shows internal and external mature fruit characteristics of ‘CULTA-T3L’, taken in the month of January 2025.

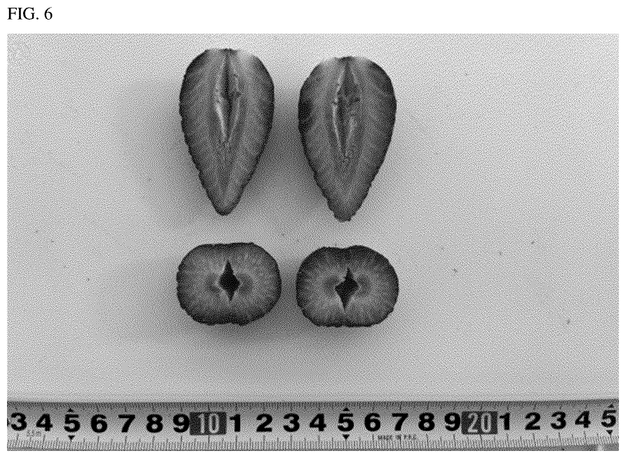

FIG. 7 shows upper and lower surfaces of leaf and leaf parts of ‘CULTA-T3L’, taken in the month of January 2025.

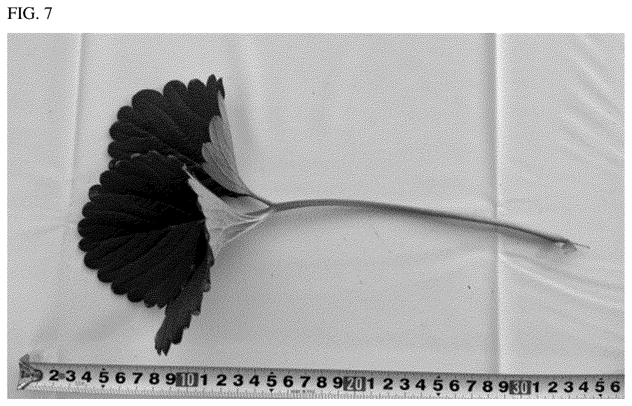

FIG. 8 shows upper surface of flower and flower parts of ‘CULTA-T3L’, taken in the month of December 2024.

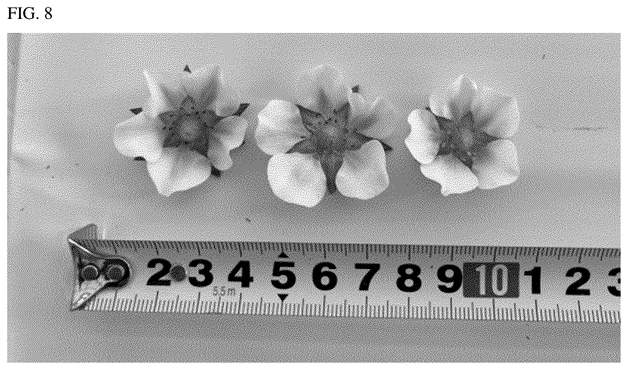

### DETAILED BOTANICAL DESCRIPTION

The new variety ‘CULTA-T3L’ has not been observed under all possible environmental conditions. The characteristics of the new variety ‘CULTA-T3L’ may vary in detail, depending upon variations in environmental factors, including weather (temperature, humidity and light intensity), day length, soil type and location. In addition, the characteristics of any comparison variety included in Table 1 of the present invention may vary in detail, depending upon variations in environmental factors, including weather (temperature, humidity and light intensity), day length, soil type and location.

In a heated greenhouse forcing culture, the planting time for the new variety ‘CULTA-T3L’ is early to mid-October. The flowering time is middle to late November. Harvesting occurs from late December to early January. The new variety ‘CULTA-T3L’ is the result of a breeding program to achieve fruit with high sugar content and high fruit firmness and plants with vigorous stolon growth. Crossbreeding was conducted in the research field in Tokyo from December 2021 to January 2022. An unnamed variety with a particularly high fruit firmness was designated as the maternal line. An unnamed variety with vigorous stolon production was designated as the paternal line. Seedling selection was conducted from October 2022 to March 2023. Secondary selection by stolon propagation was conducted from October 2023 to March 2024. Breeding of the new variety ‘CULTA-T3L’ was completed in 2024, since it was confirmed that the breeding goals to achieve characteristics of vigorous stolon production, high sugar content, and extremely high fruit firmness had been met.

The aforementioned photographs, together with the following description of the new variety ‘CULTA-T3L’, unless otherwise noted, are based on observations taken during the 2024-2025 growing season in Tokyo and Saitama, Japan. These measurements and ratings were taken from plants of ‘CULTA-T3L’ in a nursery in Saitama, Japan.

Flower measurements and characteristics are from secondary flowers unless otherwise noted. Fruit characteristics and measurements are from secondary fruit, unless otherwise noted.

The following characteristics in Table 2 describe fruit, plant, stolon, foliage, fruiting truss, flower, reproductive organs and other characteristics of the new strawberry ‘CULTA-T3L’.

| | | Supplemental | |
|---|---|---|---|
| | | information | |
| | | (measurements, | |
| Characteristic | ‘CULTA-T3L’ | etc.) | |
| Plant: growth habit | upright | | |
| Plant: density of | medium | | |
| foliage | | | |
| Plant: vigor | strong | | |
| Plant: number of | few | | |
| axillary bud | | | |
| Plant: position of | above | | |
| inflorescence in | | | |
| relation to foliage | | | |
| Plant: number of | many to very | 15 | |
| stolons | many | | |
| Leaf: size | large | 164.5 | cm² |
| Leaf: color of upper side | dark green | | |
| Leaf: blistering | medium | | |
| Leaf: glossiness | medium | | |
| Leaf: variegation | absent | | |
| Terminal leaflet: size | large | 73.6 | cm² |
| Terminal leaflet: length | moderately | 1.2 | |
| in relation to width | longer | | |
| Terminal leaflet: shape of | obtuse | | |
| base | | | |
| Terminal leaflet: margin | crenate | | |
| Terminal leaflet: profile | convex | | |
| in cross-section | | | |
| Petiole: length | long | 16.5 | cm |
| Petiole: attitude of hairs | slightly | | |
| | outwards | | |
| Stipule: intensity of | weak | | |
| anthocyanin coloration | | | |
| Inflorescence: number of | medium | 9.6 | |
| flowers | | | |
| Peduncle: length | long | 16.62 | cm |
| Pedicel: attitude of hairs | upwards | | |
| Flower: diameter | medium | 3.49 | cm |
| Flower: arrangement of | touching | | |
| petals | | | |
| Flower: size of calyx in | same size | | |
| relation to corolla | | | |
| Flower: stamen | present | | |
| Petal: length in relation | moderately | 0.89 | |
| to width | shorter | | |
| Petal: color of upper | white | | |
| side | | | |
| Fruit: size | large | 22.7 | g |
| Fruit: length in relation | much longer | 1.7 | |
| to width | | | |
| Fruit: shape | ovate | | |
| Fruit: difference in | large to very | | |
| shape of terminal | large | | |
| and other fruits | | | |
| Fruit: color | orange red | | |
| Fruit: evenness of color | slightly uneven | | |
| Fruit: glossiness | medium | | |
| Fruit: evenness of | slightly | | |
| surface | uneven | | |
| Fruit: width of band | narrow to | | |
| without achenes | medium | | |
| Fruit: neck | absent | | |
| Fruit: position of | slightly below | | |
| achenes | surface | | |
| Fruit: density of | medium | | |
| achenes | | | |
| Fruit: intensity of | medium | | |
| anthocyanin | | | |
| coloration of achenes | | | |
| Fruit: position of calyx | level with fruit | | |
| attachment | | | |
| Fruit: attitude of sepals | upwards | | |
| Fruit: diameter of calyx | slightly larger | | |
| in relation to | | | |
| diameter of fruit | | | |
| Fruit: adherence of calyx | medium | | |
| Fruit: firmness | very firm | 0.51 | kg |
| Fruit: color of flesh | orange red | | |
| (excluding core) | | | |
| Fruit: color of core | medium red | | |
| Fruit: cavity | large | | |
| Time of beginning of | medium | Nov. 25, | 2024 |
| flowering | | | |
| Time of beginning of | medium | Jan. 15, | 2025 |
| fruit ripening | | | |

| | | |
|---|---|---|
| Type of bearing | not remontant | |
| Fruit: soluble solids | high | 12.8% |
| content | | |
| Fruit: acidity | medium | 2.1% |

The measurements in Table 2 were taken from May 1, 2024 to Jan. 21, 2025. Data was collected from 10 individual plants. Fruit firmness was measured with a Fruit Firmness Tester KM-1 (Fujiwara Scientific Co., Ltd.). Brix and acidity were determined with a Brix-acidity meter for strawberry PAL-BXIACID4 (Atago Co., Ltd.).

We claim:

1. A new and distinct strawberry plant named ‘CULTA-T3L’, as herein described and illustrated by the characteristics set forth above.
